(19) United States
(12) Patent Application Publication
(10) Pub. No.: US 2025/0043607 A1
(43) Pub. Date: Feb. 6, 2025

(54) MEDICAL WASHER/DISINFECTOR

(71) Applicant: Getinge Disinfection AB, VÄXJÖ (SE)

(72) Inventors: Ola JACOBSSON, Växjö (SE); Christoffer ENGQVIST, Växjö (SE)

(21) Appl. No.: 18/716,410

(22) PCT Filed: Nov. 11, 2022
(86) PCT No.: PCT/EP2022/081583

§ 371 Date: Jun. 4, 2024

(30) Foreign Application Priority Data
Dec. 7, 2021 (EP) 21212807.8

(51) Int. Cl. E05D 15/20; A61L 2/00; E05F 15/665

(52) U.S. Cl. CPC E05D 15/20; A61L 2/00; E05F 15/665; A61L 2202/121; A61L 2202/122; A61L 2202/24; E05Y 2201/224; E05Y 2201/232; E05Y 2201/418; E05Y 2201/668

(57) ABSTRACT

The invention relates to a medical washer/disinfector, comprising a washing chamber for receiving medical articles to be washed/disinfected, an opening for accessing the interior of the washing chamber, a door which is movable from a lowered open position to a raised stop position and then to a raised closed position. The door is associated with a first slide surface. The medical washer/disinfector also comprises a carrier arrangement vertically movable by a motor, the carrier arrangement comprising a second slide surface for enabling slidable contact with the first slide surface. A spring member extending vertically upwardly from the carrier arrangement to a connection area of the door. The door is pushed upwards by the spring member until the door reaches said raised stop position. Continued upward movement of the carrier arrangement causes the spring member to become more compressed, whereby the door moves horizontally from the raised stop position to the raised closed position.

### TECHNICAL FIELD

The present disclosure relates to a medical washer/disinfector comprising a washing chamber for receiving medical articles to be washed/disinfected.

### BACKGROUND ART

Medical washers/disinfectors may typically be provided at dental clinics, surgical departments, central sterile supply departments, and similar facilities. The medical washers/disinfectors may be used for washing/disinfecting a wide range of different medical articles, such as recirculated goods, tubular instruments, surgical sets, anaesthetic items, laboratory items, bedpans, etc. In other words, a washer/disinfector should be able to handle a large variety of different type of goods, which means that sometimes a smaller load size will be processed, and at other occasions a larger load size will be processed. Medical washer/disinfectors are commonly intended to provide both cleaning of visible physical contaminants from medical devices or medical equipment, as well as a degree of disinfection, such as by hot water exposure and/or cleaning/disinfection agents.

A medical washer/disinfector is commonly configured as a cabinet having a washing chamber which is accessible through an opening that may be closed by a door or the like. The opening is normally located at a distance from the ground, such as extending from approximately the height of the waist of a person and upwards. This is a convenient working height for the staff handling the medical washer/disinfector. Articles may, for instance, be brought to the medical washer/disinfector by means of a cart from which they are unloaded and loaded into the medical washer/disinfector. Another loading possibility is using a conveyor belt or the like which transports the articles into the medical washer/disinfector.

As explained above, medical washers/disinfector may be provided at various different facilities. Sometimes the available space for installing a medical washer/disinfector is rather limited. Such limited available space puts a limitation on the dimension of the medical washer/disinfector. Not only is the dimension relevant to consider in a closed state, but also in an open state, since the available displacement of the door from its closed position to its open position may also be restricted by the facility at which the medical washer/disinfector is to be installed. It would therefore be desirable to provide a medical/disinfector which takes such limited space facilities into consideration.

### SUMMARY OF THE INVENTION

An object of the present disclosure is to provide a medical washer/disinfector which at least partly alleviates the drawbacks of the prior art and which enables installation in facilities having limited available space. This and other objects, which will become apparent in the following, are accomplished by a method as defined in claim 1. Exemplary embodiments are presented in the dependent claims.

The general inventive concept is based on the realization that the distance below the level of the opening to the washing chamber may be utilized for displacement of the door to its open position. By making use of this space below the opening, no substantial additional space is required for opening the door, as opposed to swinging the door open by a pivoting motion, or raising the door above the top of the medical washer/disinfector which needs a large distance to the ceiling of the facility. Furthermore, it has been realized that since a vertical movement of the door should suitably be performed with a small play between the door and the material surrounding the opening (to avoid friction/chafing) an upwardly directed force provided by a motor to raise the door may also be used for closing such play. In particular, the inventor has realized that the upwards force caused by the motor may be used to raise the door and at the appropriate raised position the continued upward force is transferred into a horizontal closing force. Thus, even though a continued upwards force is provided by the motor, the door will not be raised too high. The general inventive concept and exemplary embodiments thereof will now be discussed in more detail.

According to a first aspect of the present disclosure, there is provided a medical washer/disinfector, comprising:
a washing chamber for receiving medical articles to be washed/disinfected,
an opening for accessing the interior of the washing chamber,
a door which is movable
   from a lowered open position, enabling medical articles to be inserted into or retrieved from the washing chamber through the opening,
   and then to a raised stop position, in which the door is located in front of the opening but positioned laterally spaced apart from the opening,
   and then to a raised closed position, in which the door is positioned against the opening and seals the opening,
   wherein the door also passes through the raised stop position when transitioning from the raised closed position back to the lowered open position, the door comprising of being fixedly connected to a first slide surface,
a motor,
a carrier arrangement vertically movable by the motor, the carrier arrangement comprising a second slide surface for enabling slidable contact with the first slide surface,
a spring member connected to the door at a connection area, the spring member being in contact with the carrier arrangement and extending vertically upwardly from the carrier arrangement to said connection area,
   wherein when the door is in the lowered open position and the motor moves the carrier arrangement upwards, the door is pushed upwards by the spring member until the door reaches said raised stop position at which point further upwards movement of the door is prevented,
   wherein the pushing upwards by the spring member prevents sliding motion between the first and second slide surfaces during the movement of the door from the lowered open position to the raised stop position,
   wherein, after the door has reached the raised stop position, continued upward movement of the carrier arrangement causes the spring member to become more compressed, thereby enabling the first slide surface to slide along the second slide surface, whereby the door moves horizontally from the raised stop position to the raised closed position due to the continued upward movement of the carrier arrangement.

By opening the door downwardly to a lowered position, and raising the door upwardly and allowing the door to be subject to the upwardly directed force stemming from the motor but transferring it to a horizontal closing motion, a compact yet efficient medical washer/disinfector is obtainable. The spring member will be in a substantial steady state during the upwards motion of the door pushing the door upwardly and keeping it at a substantially fixed lateral distance from the opening. Thus, the spring member keeps the door from self-locking during the movement from the lowered open position to the raised stop position (and also during the reverse movement, i.e. from the raised stop position to the lowered open position). However, when the door is prevented from moving further upwardly, the continued upwards movement of the carrier arrangement results in that the carrier arrangement approaches the connection area of the door. Due to this closing-in of the carrier arrangement, the spring member (extending between the carrier arrangement and the connection area door) becomes more compressed, and cannot keep the door laterally spaced from the opening. Instead, the door will now by means of the first and second slide surfaces move horizontally to close the opening.

According to at least one exemplary embodiment, at least one of the first and second slide surfaces is inclined such that it is at a higher vertical level distally to the washing chamber and at a lower vertical level proximally to the washing chamber. By having at least one slide surface inclined an efficient horizontal motion will be achieved as the spring becomes gradually more and more compressed as the motor continues driving the carrier arrangement upwardly. Suitably both the first and second slide surfaces are inclined, and suitably at the same angle of inclination, thereby providing for a larger interface between the slide surfaces. The inclination may suitably be straight, such as forming an angle of inclination of 30°-60° relative to a vertical extension. For instance, for an angle of inclination of 45°, when the door has reached the raised stop position, the door would move one unit length (e.g. 1 mm) in the horizontal direction for each unit length (e.g. 1 mm) that the carrier arrangement continues to move upwards in the vertical direction. It should be understood, that other inclinations of the first and/or second slide surfaces are conceivable, such as for instance, a curved inclination.

According to at least one exemplary embodiment, the medical washer/disinfector comprises at least one slide member fixedly connected to a lateral side of the door, wherein the slide member comprises said first slide surface, wherein said first slide surface is inclined such that it is at a higher vertical level distally to the washing chamber and at a lower vertical level proximally to the washing chamber. By having a slide member fixed to the door is convenient from a manufacturing perspective, as it may be easier to provide said first slide surface on a separate member than on the door itself. The inclination of the first slide surface on the slide member may have anyone of the previously discussed configurations, or in any other appropriate configuration.

According to at least one exemplary embodiment, the carrier arrangement comprises a first carrier element and a second carrier element, the first carrier element being located above and vertically spaced apart from the second carrier element, wherein the spring member is in contact with the first carrier element and extending vertically upwardly from the first carrier element, wherein an upper side of the second carrier element presents said second slide surface. By separating the carrier arrangement into at least a first and a second carrier element, the different functionalities may be better taken into account. The second slide surface on the second carrier element may be designed independently of the surface on the first carrier element which may be design for appropriately supporting the spring member. For exemplary embodiments that include the previously mentioned slide member that is fixedly connected to the door, such a slide member may suitably be arranged between the first and second carrier elements.

According to at least one exemplary embodiment, the medical washer/disinfector comprises a gasket around the opening, wherein the door seals against the gasket when it is in its raised closed position. The gasket provides an improved sealing interface against the door.

According to at least one exemplary embodiment, said spring member is a first spring member, the medical washer/disinfector further comprising a second spring member, wherein the second spring member is configured to accumulate elastic potential energy when the door is moved from the raised stop position to the raised closed position, wherein when the door is in the raised closed position and the motor starts to move the carrier arrangement downwardly, the second spring member releases the elastic potential energy to separate the door from the gasket. This is advantageous since the gasket may provide some degree of “stickiness”, which may result in that the door does not separate well enough from the gasket when moving from the raised closed position to the raised stop position and then to its lowered open position. The second spring member will thus give the door an extra push to make sure there is a play between the gasket and the door during the vertical downwards movement of the door.

According to at least one exemplary embodiment, said second spring member comprises a turnable wheel and a spring portion, wherein the turnable wheel is connected to the carrier arrangement and wherein the spring portion interconnects the turnable wheel with the door, wherein when the door is moved from the raised stop position to the raised closed position, the spring portion pulls and turns the turnable wheel. This provides an efficient mechanism for ensuring the above mentioned separation of the door from the gasket.

According to at least one exemplary embodiment, the motor comprises a self-locking gearbox, thereby preventing the carrier arrangement from moving uncontrollably downwards when the door is in the raised closed position. Thus, the risk of the door not sealing properly during a washing/disinfecting process is avoided. It should, however, be understood that there are other conceivable alternatives for preventing such uncontrollable downwards motion. For instance, separate locking elements may be provided for engaging with the carrier arrangement.

As has already been understood from the above discussion, in order to avoid unnecessary friction during the vertical movement of the door, there should suitably be a play between the door and the area around the opening during such vertical movement. This is at least partly reflected in the following exemplary embodiment, according to which, when the door is moved from the lowered open position to the raised stop position, the door substantially floats on the spring member so that the second slide surface substantially does not press against or slide along the first slide surface.

Similarly, as has already been understood from the above discussion, when the door is moved upwardly and reaches the raised stop position, the continued upward vertical movement of the carrier arrangement will instead be transferred into a horizontal movement of the door. This is at least partly reflected in the following exemplary embodiment, according to which when the door is moved from the raised stop position to the raised closed position, vertical movement of the door is substantially prohibited, the spring member is compressed, and upwards pushing of the second slide surface against the first slide surface causes sliding by the first slide surface which moves the door horizontally into the raised closed position.

According to at least one exemplary embodiment, the medical washer/disinfector further comprises:
a limit switch, and
a shuttle configured to move in the opposite vertical direction relative to the carrier arrangement, and configured to engage and activate the limit switch during its downwards vertical movement when the door has moved from the raised stop position to the raised closed position,
   wherein activation of the limit switch stops the carrier arrangement from moving further vertically upwards and thus stops the door from moving further towards, or pressing further against, the area around the opening,
   wherein the vertical position of the limit switch is adjustable, thereby enabling adjustment of the sealing pressure of the door.

This is advantageous as it may be desirable to have different sealing pressures for different applications.

As can be understood from the previous discussion, the force provided by one motor is enough to achieve both the vertical movement of the door from the lowered open position to the raised stop position and the horizontal movement of the door from the raised stop position to the raised closed position. Thus, according to at least one exemplary embodiment, the medical washer/disinfector comprises only one motor for moving the door.

The motor disclosed herein may suitably be controlled by a control unit. The control unit may receive input commands from a user interface, and control the motor based on such input commands. The user interface may, for instance, be a control panel on the medical washer/disinfector and/or a remotely located input device (such as a remote computer or cell phone).

The control unit may include a microprocessor, microcontroller, programmable digital signal processor or another programmable device. The control unit may also, or instead, include an application specific integrated circuit, a programmable gate array or programmable array logic, a programmable logic device, or a digital signal processor. Where it includes a programmable device such as the microprocessor, microcontroller or programmable digital signal processor mentioned above, the processor may further include computer executable code that controls operation of the programmable device. The control unit is preferably configured for use with medical washer/disinfectors, and provided with electronic instructions to perform and control the opening and closing of the door.

According to a second aspect of this disclosure, there is provided a method for operating a medical washer/disinfector of the first aspect, including any exemplary embodiment thereof. The method comprises:
moving the door from said lowered open position to the raised stop position by pushing the door upwards by means of said spring member until the door reaches the raised stop position, and then
moving the door from the raised stop position to the raised closed position by pressing the door in a horizontal direction by means of the carrier arrangement.

The reader will understand that the various features and exemplary embodiments discussed in connection with the medical washer/disinfector of the first aspect may be readily implemented as corresponding exemplary embodiments of the method of the second aspect.

Generally, all terms used in the claims are to be interpreted according to their ordinary meaning in the technical field, unless explicitly defined otherwise herein. All references to “a/an/the element, apparatus, component, means, step, etc.” are to be interpreted openly as referring to at least one instance of the element, apparatus, component, means, step, etc., unless explicitly stated otherwise. The steps of any method disclosed herein do not have to be performed in the exact order disclosed, unless explicitly stated. Further features of, and advantages with, the present invention will become apparent when studying the appended claims and the following description. The skilled person realizes that different features of the present invention may be combined to create embodiments other than those described in the following, without departing from the scope of the present inventive concept.

### BRIEF DESCRIPTION OF THE DRAWINGS

FIG. 1 illustrates a medical washer/disinfector in an open state, in accordance with at least one exemplary embodiment of the invention.

FIG. 2 illustrates the medical washer/disinfector of FIG. 1 in a closed state.

FIG. 3 illustrate certain components involved in effecting a change from the open state to the closed state of the medical washer/disinfector, in accordance with at least one exemplary embodiment of the invention.

FIG. 4 is a zoomed-in view of FIG. 3, showing the door in its raised stop position.

FIG. 5 corresponds to the view in FIG. 4 but showing the door in it its raised closed position.

FIG. 6 illustrates a detailed view of a mechanism for facilitating the door to return from its raised closed position to its raised stop position.

FIG. 7 illustrates a detailed view of a mechanism enabling adjustment of the sealing pressure.

FIG. 8 illustrates a detailed view of the location of the motor.

### DETAILED DESCRIPTION

The present inventive concept will now be described more fully hereinafter with reference to the accompanying drawings, in which certain aspects of the inventive concept are shown. The inventive concept may, however, be embodied in many different forms and should not be construed as limited to the embodiments and aspects set forth herein; rather, the embodiments are provided by way of example so that this disclosure will be thorough and complete, and will fully convey the scope of the inventive concept to those skilled in the art. Accordingly, it is to be understood that the present inventive concept is not limited to the embodiments described herein and illustrated in the drawings; rather, the skilled person will recognize that many changes and modifications may be made within the scope of the appended claims. Like reference numerals refer to like elements throughout the description.

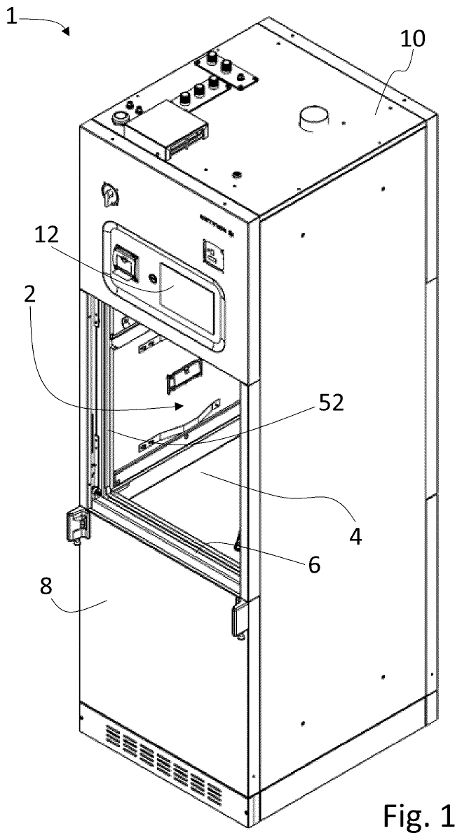

FIG. 1 illustrates a medical washer/disinfector 1 in an open state, in accordance with at least one exemplary embodiment of the invention. The medical washer/disinfector 1 comprises a washing chamber 2 for receiving medical articles to be washed/disinfected. An opening 4 is provided for accessing the interior of the washing chamber 2. The medical washer/disinfector 1 will normally be standing on a floor in a facility, and the opening 4 is located at an elevated level from the floor to allow for a convenient working height for a user. The medical washer/disinfector 1 is also provided with a movable door 6. In FIG. 1 only a top surface of the door 6 is discernible. The rest of the door 6 is located behind a front panel 8 of the medical washer/disinfector 1. Thus, in this illustrated open state of the medical washer/disinfector 1, the door 6 is in a lowered open position, enabling medical articles to be inserted into or retrieved from the washing chamber 2 through the opening 4. This is advantageous as the vertical space below the opening 4 is needed anyway for providing a convenient working height, and the inventive concept makes use of this available vertical space by allowing the door 6 to move downwardly for giving an operator access to the washing chamber 2. If the door 6 would instead have opened upwardly, it would need to extend past the top 10 of the medical washer/disinfector 1, which may not be practically possible when installed in facilities having a low ceiling.

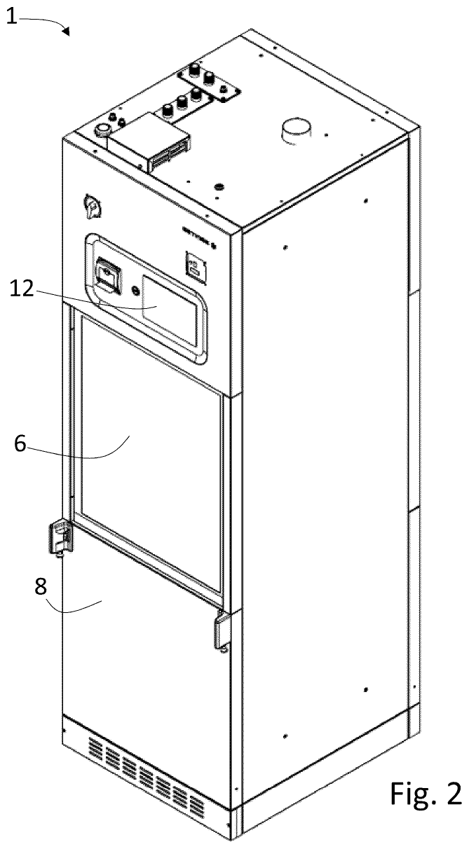

The door 6 is movable from the lowered open position to a raised stop position in which the door 6 is located in front of the opening 4 but positioned laterally spaced apart from the opening, and then to a raised closed position in which the door 6 is positioned against the opening 4 and seals the opening 4. This raised closed position is illustrated in FIG. 2. After a completed washing/disinfection program, the door 6 is moved in the reverse order. Thus, the door 6 will move through the raised stop position when transitioning from the raised closed position (FIG. 2) back to the lowered open position (FIG. 1).

The displacement of the door 6 may be activated by an operator, either directly or indirectly (for example by selecting a washing/disinfecting program). The operator may provide instructions to the medical washer/disinfector 1 via a user interface 12. In FIGS. 1 and 2, a user interface 12 is exemplified in the form of a control panel on the medical washer/disinfector 1, however, other local or remote user interfaces are conceivable alternatives or supplements.

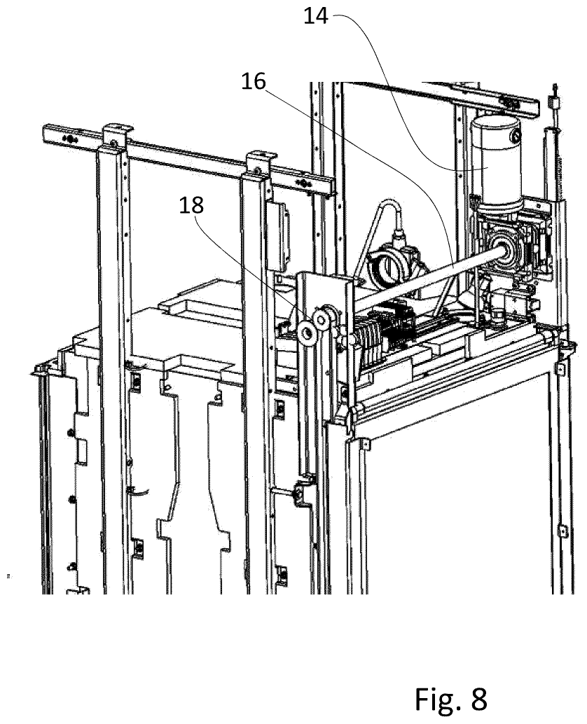

The displacement of the door 6 is powered by a motor. An example of such a motor 14 is illustrated in FIG. 8. The rotary motion of the motor 14 may be transmitted/translated to a rotary motion of a shaft 16 connected to one or more with pulleys 18 for moving a band, belt, chain, or the like to which a carrier arrangement is connected. The motor 14 may suitably comprise a self-locking gearbox, thereby preventing the carrier arrangement from moving uncontrollably downwards when the door 6 is in the raised closed position.

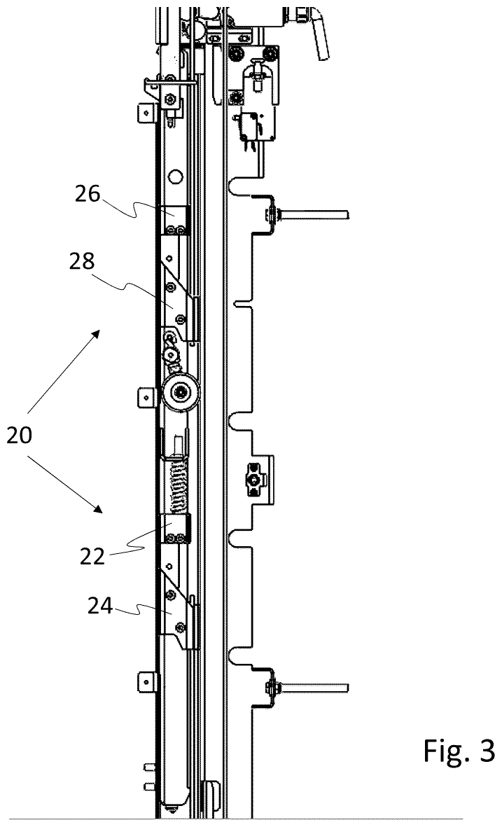

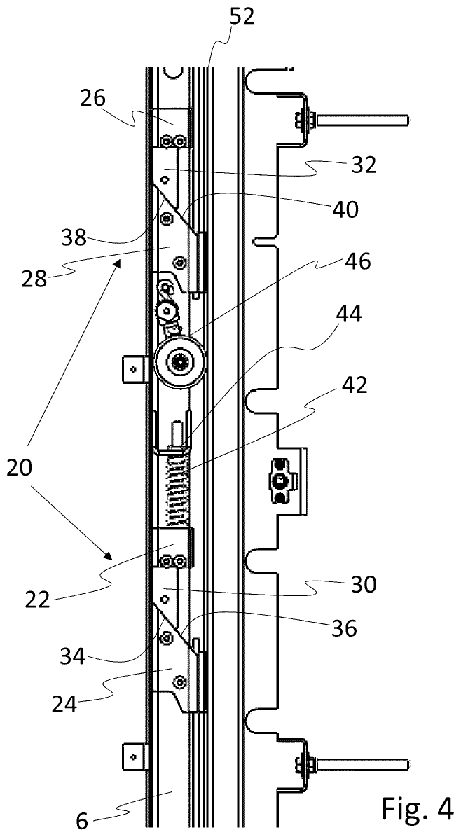

FIG. 3 illustrates an example of such a carrier arrangement 20. The view of FIG. 3 is a detailed side view from the right side of FIG. 1, as seen from within the outer visible housing of FIG. 1. The carrier arrangement 20 is vertically movable by the motor 14 (via the shaft 16, pulleys 18, band, etc.), though not illustrated in FIGS. 3. In the illustrated exemplary embodiment, the carrier arrangement 20 comprises a first carrier element 22 and a second carrier element 24, the first carrier element 22 being located above and vertically spaced apart from the second carrier element 24. The carrier arrangement 20 may further comprise a third carrier element 26 and a fourth carrier element 28, the third carrier element 26 being located above and vertically spaced apart from the fourth carrier element 28. The fourth carrier element 28 is in its turn located above and vertically spaced apart from the first carrier element 22. The carrier arrangement 20 with its first, second, third and fourth carrier elements 22, 24, 26 and 28, respectively, can be seen more clearly in the zoomed-in view of FIG. 4.

The door 6 may comprise a first slide surface, or as illustrated in FIG. 4, the door 6 may comprise a slide member 30 fixedly connected to a lateral side of the door 6 (i.e. not the front, back, top or bottom sides of the door) and that slide member 30 may comprise such a first slide surface 34. The slide member 30 is a lower slide member 30, as there is also provided an upper slide member 32. The lower slide member 30 is located between the first carrier element 22 and the second carrier element 24. The upper slide member 32 is located between the third carrier element 26 and the fourth carrier element 28. Suitably, the opposite lateral side of the door 6 has a corresponding configuration with slide members and carrier elements. Nevertheless, it should be understood that the number of carrier elements may be different than what has now been illustrated. For instance, in some exemplary embodiments the third and fourth carrier elements 26, 28, as well as the upper slide member 32 may be omitted, while in other exemplary embodiments, there may be provided additional carrier elements and slide member(s).

According to the general inventive concept, the carrier arrangement 20 comprises a second slide surface for enabling slidable contact with the first slide surface 34. In this exemplary embodiment, the second carrier element 24 presents such a second slide surface 36 forming an interface with the first slide surface 34 of the lower slide member 30. In particular, it is the upper side of the second carrier element 24 that presents said second slide surface 36 which mates with the first slide surface 34, which is located at a lower side of said lower slide member 30. According to at least some exemplary embodiments, such as the one illustrated in FIG. 4 (but also in others), the second slide surface 36 has a longer extension than the first slide surface 34. In particular, the second slide surface 36 may have a longer extension than the first slide surface 34 as seen in the sliding direction.

Similarly to the second carrier element 24, the fourth carrier element 28 has a corresponding second slide surface 40 mating with the first slide surface 38 of the upper slide member 32.

As can be seen in FIG. 4, each one of the first and second slide surfaces 34, 36, 38, 40 is inclined such that it is at a higher vertical level distally to the washing chamber and at a lower vertical level proximally to the washing chamber. This enables a good sliding of the first slide surfaces 34, 38 along the respective second slide surfaces 36, 40. It should, however, be understood, that a similar sliding motion (directionwise) could also be achieved, if only one of the mating first and second slide surfaces 34, 36, 38, 40 (in each pair) would have such an inclination.

The medical washer/disinfector 1 also comprises a spring member, which in this exemplary embodiment is illustrated as a first spring member 42 (see FIG. 4), which is connected to the door 6 at a connection area 44. The first spring member 42 is in contact with the carrier arrangement 20. In this particular exemplary embodiment, the first spring member 42 is in contact with the first carrier element 22 of the carrier arrangement 20. The first spring member 42 extends vertically upwardly from the first carrier element 22 to said connection area 44.

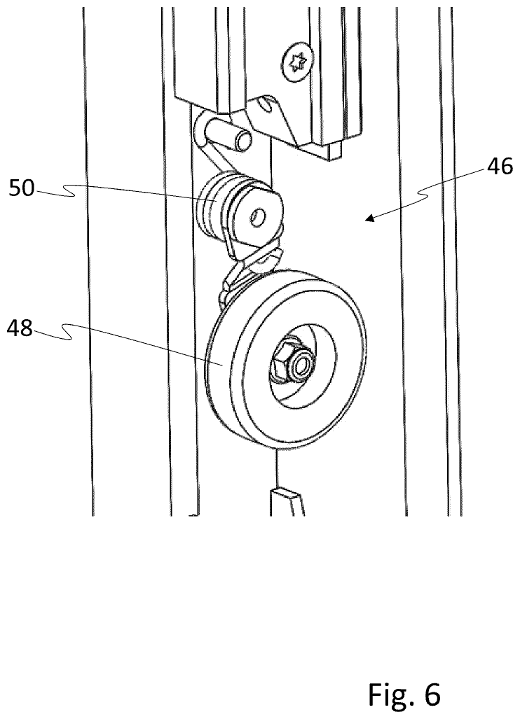

The medical washer/disinfector 1 also comprises a second spring member 46 (see for example FIGS. 4 and 6). As best seen in FIG. 6, the spring member 46 comprises a turnable wheel 48 and a spring portion 50. The turnable wheel 48 is connected to the carrier arrangement 20 and the spring portion 50 interconnects the turnable wheel 48 with the door 6. Although not seen in the drawings, the carrier arrangement 20 may suitably comprise a beam connected to the carrier elements, suitably extending all the way from the second carrier element 24 to the third carrier element 26 (and being located closer to the viewer of the drawing figure than the plane currently viewed in FIG. 4). Such a beam may, for instance, be fastened to one or more of the carrier elements 22, 24, 26, 28 by means of the illustrated bolts shown in the carrier elements 22, 24, 26, 28. Such a beam will thus follow the motions of the carrier elements 22, 24, 26, 28. The turnable wheel 48 of the second spring member 46 may suitably be connected to such a beam of the carrier arrangement 20. In the presently illustrated exemplary embodiment, the second spring member 46 is located between first spring member 42 and the fourth carrier element 28. It should, however, be understood that other locations of the second spring member 46 are also conceivable while still performing its intended function, which will be explained further below in this disclosure.

In the illustrated exemplary embodiment, the medical washer/disinfector 1 further comprises a gasket 52 (see e.g. FIGS. 1 and 4) around the opening 4, wherein the door 6 is configured to seal against the gasket 52 when the door 6 is in its raised closed position.

In operation, when the door 6 is in the lowered open position (FIG. 1) and the motor 14 (FIG. 8) moves the carrier arrangement 20 (FIG. 4) upwards, the door 6 is pushed upwards by the first spring member 42. Despite the inclined interface between the mating first slide surfaces 34, 38 (facing downwardly) and the respective second slide surface 36, 40 (facing upwardly), the door 6 remains substantially stationary in the horizontal direction during its upward motion, i.e. the horizontal separation between the door 6 and the opening 4 remains substantially the same during the upwards motion of the door 6. This is due to the first spring member 42. The first spring member 42 prevents sliding motion between the first and second slide surfaces 34, 36, 38, 40 during the movement of the door 6 from the lowered open position to the raised stop position illustrated in FIG. 4. Thus, the first spring member 42 is strong enough to withstand the gravitational effect on the door 6. Without the first spring member 42 the door 6 would become self-closing, as the slide members 30, 32 on the door 6 would, due to gravitation, slide along the respective carrier elements 36, 40. Accordingly, during the movement of the door 6 from the lowered open position to the raised stop position of FIG. 4, the slide members 30, 32 will be located at the relatively distal position shown in FIG. 4, i.e. distal relative to the opening 4 and the surrounding gasket 52.

From the above it can be understood that the door 6 can be regarded as substantially floating on the first spring member 42. This floating will continue throughout the upwards motion of the door 6. The door 6 is pushed upwards by the first spring member 42 until the door 6 reaches the raised stop position (shown in FIG. 4) at which point further upwards movement of the door 6 is prevented.

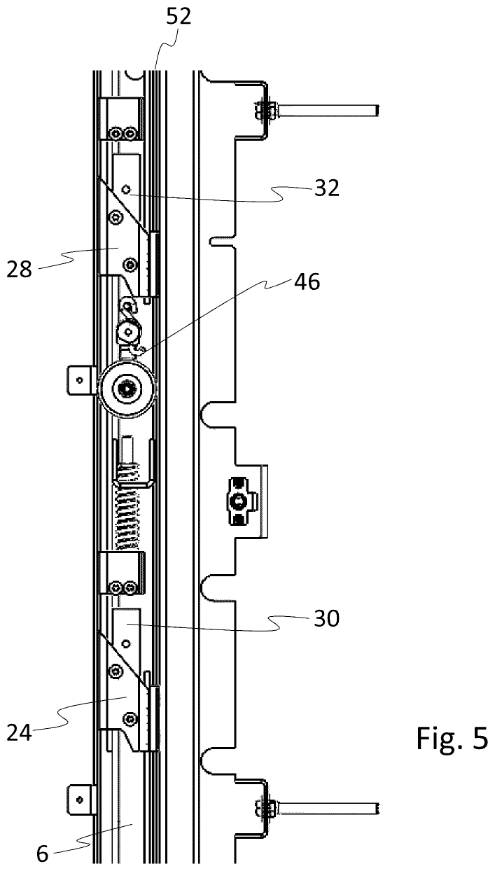

After the door 6 has reached the raised stop position, continued upward movement of the carrier arrangement 20 causes the first spring member 42 to become more compressed, thereby enabling the first slide surfaces 34, 38 of the slide members 30, 32 to slide along the respective mating second slide surfaces 36, 40 of the second and fourth carrier elements 24, 28. This causes the door 6 to move horizontally from the raised stop position (FIG. 4) to the raised closed position (FIG. 5) due to the continued upward movement of the carrier arrangement 20. In other words, when the door 6 is moved from the raised stop position (FIG. 4) to the raised closed position (FIG. 5), vertical movement of the door 6 is substantially prohibited, the first spring member 42 is compressed, and upwards pushing of the second slide surfaces 36, 40 against the first slide surfaces 34, 38 causes sliding by the first slide surfaces 34, 38 which moves the door 6 horizontally into the raised closed position illustrated in FIG. 5.

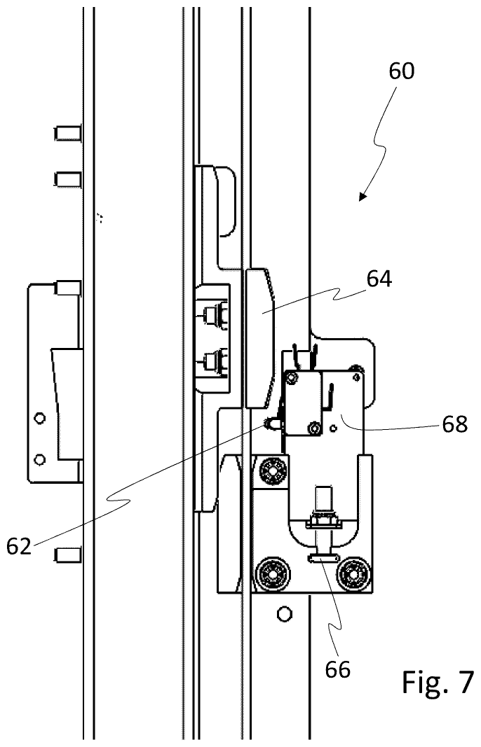

As can be seen in FIG. 5, the slide members 30, 32 have now moved along the second and fourth carrier elements 24, 28, respectively, in comparison to position of the slide members 30, 32 in FIG. 4. In the raised closed position of FIG. 5, the slide members 30, 32 are located at a relatively proximal position, i.e. proximal relative to the opening and the surrounding gasket 52. The medical washer/disinfector 1 may suitably be configured to allow an adjustable sealing pressure of the door 6 against the gasket 52. Thus, for the raised closed position illustrated in FIG. 5, the sealing pressure may suitably be selectable. This will now be exemplified in connection with FIG. 7.

FIG. 7 illustrates a detailed view of a part of the medical washer/disinfector, for example a lower part thereof, which is not visible in the other drawing figures. The medical washer/disinfector comprises an actuator mechanism 60 for enabling adjustment of the sealing pressure. The actuator mechanism 60 comprises a limit switch 62 and a shuttle 64. The shuttle 64 is configured to move in the opposite vertical direction relative to the carrier arrangement. Thus, when the carrier arrangement moves upwards, the shuttle 64 moves downwards, and when the carrier arrangement moves downwards, the shuttle 64 moves upwards. The shuttle 64 is configured to engage and activate the limit switch 62 during its downwards vertical movement when the door has moved from the raised stop position to the raised closed position. Activation of the limit switch 62 stops the carrier arrangement from moving further vertically upwards and thus stops the door from being pressed (by the second slide surfaces) further towards the opening and the surrounding gasket. The vertical position of the limit switch 62 is adjustable. In FIG. 7, this is illustrated by means of a turnable screw 66, which via a plate 68 moves the limit switch 62 up or down (depending on the direction in which you turn the screw 66). Thus, by setting a desired vertical position of the limit switch 62, you can decide how much more the carrier arrangement may continue to move upwards and thus continue to press the door against the gasket by (means of the slide surface interfaces) after the door has already reached the raised position. In this way the sealing pressure may be selected.

It has been noted that the selected sealing pressure may also affect the ability of the door to separate from the gasket when the door is to be opened. In order to facilitate proper separation of the door from the gasket and enabling the door to reach its laterally spaced apart raised stop position before being lowered into the lowered open position, the above mentioned second spring member has been provided.

Turning tow FIG. 6, the second spring member 46 is configured to accumulate elastic potential energy when the door is moved from the raised stop position to the raised closed position. When the door is in the raised closed position and the motor starts to move the carrier arrangement downwardly, the second spring member 46 releases the elastic potential energy to separate the door from the gasket. More specifically, when the door is moved from the raised stop position (FIG. 4) to the raised closed position (FIG. 5), the spring portion 50 pulls and turns the turnable wheel 48, as can be understood when comparing the different states of the second spring member 46 shown in FIGS. 4 and 5. In this way, the energy is accumulated in the spring portion 50 and later released to assist in separating the door in the horizontal direction from the gasket 52.

This disclosure includes medical washer/disinfectors. This disclosure also includes controllers/control units (comprising electronics and electronic instructions) for controlling washer/disinfectors as described herein. It is also understood from this disclosure, that it further includes methods of operating and controlling washer/disinfectors, such as methods of opening and closing a door of a medical washer/disinfector. It should be understood that various features and methods disclosed herein are contemplated and disclosed in their various combinations and sub-combinations.

1-11. (canceled)

12. A medical washer/disinfector, comprising:
a washing chamber receiving medical articles to be washed/disinfected;
an opening accessing an interior of the washing chamber;
a door configured to be movable: from (a) a lowered open position, enabling medical articles to be inserted into or retrieved from the washing chamber through the opening, and then to (b) a raised stop position, in which the door is located in front of the opening but positioned laterally spaced apart from the opening, and then to (c) a raised closed position, in which the door is positioned against the opening and seals the opening, wherein the door also passes through the raised stop position when transitioning from the raised closed position back to the lowered open position, the door comprising or being fixedly connected to a first slide surface;
a motor;
a carrier arrangement vertically movable by the motor, the carrier arrangement comprising a second slide surface configured to enable slidable contact with the first slide surface; and
a spring member connected to the door at a connection area, the spring member being in contact with the carrier arrangement and extending vertically upwardly from the carrier arrangement to the connection area,
wherein when the door is in the lowered open position and the motor moves the carrier arrangement upwards, the door is pushed upwards by the spring member until the door reaches the raised stop position at which point further upwards movement of the door is prevented,
wherein the pushing upwards by the spring member prevents sliding motion between the first and second slide surfaces during the movement of the door from the lowered open position to the raised stop position,
wherein, after the door has reached the raised stop position, continued upward movement of the carrier arrangement causes the spring member to become more compressed, thereby enabling the first slide surface to slide along the second slide surface, whereby the door moves horizontally from the raised stop position to the raised closed position due to the continued upward movement of the carrier arrangement.

13. The medical washer/disinfector of claim 12, wherein at least one of the first and second slide surfaces is inclined such that it is at a higher vertical level distally to the washing chamber and at a lower vertical level proximally to the washing chamber.

14. The medical washer/disinfector of claim 12, further comprising:
at least one slide member fixedly connected to a lateral side of the door,
wherein the slide member comprises the first slide surface,
wherein the first slide surface is inclined such that it is at a higher vertical level distally to the washing chamber and at a lower vertical level proximally to the washing chamber.

15. The medical washer/disinfector of claim 12, wherein the carrier arrangement comprises a first carrier element and a second carrier element, the first carrier element being located above and vertically spaced apart from the second carrier element, wherein the spring member is in contact with the first carrier element and extending vertically upwardly from the first carrier element, wherein an upper side of the second carrier element presents the second slide surface.

16. The medical washer/disinfector of claim 12, wherein when the door is moved from the lowered open position to the raised stop position, the door substantially floats on the spring member so that the second slide surface substantially does not press against or slide along the first slide surface.

17. The medical washer/disinfector of claim 16, wherein when the door is moved from the raised stop position to the raised closed position, vertical movement of the door is substantially prohibited, the spring member is compressed, and upwards pushing of the second slide surface against the first slide surface causes sliding by the first slide surface which moves the door horizontally into the raised closed position.

18. The medical washer/disinfector of claim 12, further comprising:
a gasket situated around the opening,
wherein the door seals against the gasket when it is in its raised closed position.

19. The medical washer/disinfector of claim 18, wherein the spring member is a first spring member and wherein the medical washer/disinfector is further comprising:
a second spring member configured to accumulate elastic potential energy when the door is moved from the raised stop position to the raised closed position,
wherein when the door is in the raised closed position and the motor starts to move the carrier arrangement downwardly, the second spring member releases the elastic potential energy to separate the door from the gasket.

20. The medical washer/disinfector of claim 19, wherein the second spring member comprises a turnable wheel and a spring portion, wherein the turnable wheel is connected to the carrier arrangement, wherein the spring portion interconnects the turnable wheel with the door, sand wherein when the door is moved from the raised stop position to the raised closed position, the spring portion pulls and turns the turnable wheel.

21. The medical washer/disinfector of claim 12, wherein the motor comprises a self-locking gearbox, thereby preventing the carrier arrangement from moving uncontrollably downwards when the door is in the raised closed position.

22. The medical washer/disinfector of claim 12, further comprising:
a limit switch; and
a shuttle configured to move in an opposite vertical direction relative to the carrier arrangement and configured to engage and activate the limit switch during its downwards vertical movement when the door has moved from the raised stop position to the raised closed position,
wherein activation of the limit switch stops the carrier arrangement from moving further vertically upwards and thus stops the door from moving further towards or pressing further against the opening, and
wherein the vertical position of the limit switch is adjustable, thereby enabling adjustment of a sealing pressure of the door.
